(19) United States
(12) Patent Application Publication
(10) Pub. No.: US 2019/0290144 A1
(43) Pub. Date: Sep. 26, 2019

(54) SINGLE POINT NON-OCCLUDING BLOOD PRESSURE SENSOR

(71) Applicant: Nathanael Schwab, Santa Cruz, CA (US)

(72) Inventor: Nathanael Schwab, Santa Cruz, CA (US)

(21) Appl. No.: 16/297,671

(22) Filed: Mar. 10, 2019

Related U.S. Application Data
(60) Provisional application No. 62/647,909, filed on Mar. 26, 2018.

(51) Int. Cl. A61B 5/024; A61B 5/021; A61B 5/026

(52) U.S. Cl. CPC A61B 5/02416; A61B 5/02007; A61B 5/0261; A61B 5/02108

(57) ABSTRACT

A blood pressure monitor includes a finger clip portion and a host portion. Multiple sensors are incorporated into the clip and obtain optical and mechanical response data from arteries in the finger. The clip urges the sensors against the skin of the finger without occluding blood flow. A processor in the host portion is operable to compute systolic and diastolic blood pressure based on extracted features from the sensed data and individual characteristics of the user. Related methods are also described.

### CROSS-REFERENCE TO RELATED APPLICATIONS

This application claims the benefit of U.S. Provisional Application No. 62/647,909, filed Mar. 26, 2018, the entire contents of which are incorporated herein by reference in their entirety for all purposes.

### BACKGROUND OF THE INVENTION

The invention relates to blood pressure measurement, and more particularly, to blood pressure measurement without occluding a blood vessel, namely, cuffless.

Blood pressure determination is a commonly used clinical parameter for diagnosis and monitoring of cardiac conditions. Currently blood pressure is determined using a sphygmomanometer which inflates a cuff to cut off blood flow and measure the pressure at which blood flow returns. This method is not ideal because it is uncomfortable, time consuming, and inconvenient. Creating a cuffless blood pressure device would alleviate these disadvantages.

There have been attempts to make a cuffless blood pressure device that uses pulse transit time (PTT). However, PTT is not reliable because PTT requires taking data at multiple points on the body. Additionally, PTT is not a direct measurement of pressure and thus is not completely accurate.

Examples of cuffless blood pressure patents that use pulse transit time to determine pressure include U.S. Pat. Nos. 6,723,054; 7,674,231; and 6,331,162.

Research literature also describes attempts to estimate blood pressure using photoplethysmograms (PPGs) extracted from the patient. Despite noticeable subjective differences in PPG waveforms between people with higher/lower blood pressure, attempts to empirically determine blood pressure from these differences only slightly outperforms estimation using only the height of patient. (R2=0.826 for estimation using PPG R2=0.703 for estimation using height) (Hangsik Shin and Se Dong Min, 2017, et al). This shows that the PPG morphology does contain information useful in estimating blood pressure but is not reliable enough on its own to meet the standards necessary for clinical use.

The modern theoretical understanding of arterial wall mechanics also supports this conclusion. The elastic response of arteries to blood flow is highly nonlinear: following a viscoelastic stress strain relationship. There are also hidden variables relating to the effect of surrounding tissues to the artery such as collagen, elastin, and smooth muscle fibers. This means that although there is a causal relationship between the blood pressure and volumetric flow of blood (which can be determined by the PPG), that relationship is ill-defined.

Notwithstanding the above, an apparatus and related method that overcomes the above mentioned challenges is desirable.

### SUMMARY OF THE INVENTION

A blood pressure monitor includes a finger clip portion and a host portion. Multiple sensors are incorporated into the clip and obtain optical and mechanical response data from arteries in the finger. In embodiments, the mechanical response sensor is a piezo sensor.

The clip urges the sensors against the skin of the finger without occluding blood flow. A processor in the host portion is operable to compute systolic and diastolic blood pressure based on extracted features from the sensed data and individual characteristics of the user.

In another embodiment, a method for automatically determining blood pressure of a person comprises: obtaining optical data by placing an optical sensor against the skin of the person; obtaining mechanical response data by placing a mechanical response sensor against the skin without occluding a blood vessel under the skin; and computing on a processor the blood pressure of the person based on the optical data and the mechanical response data.

In embodiments, prior to the computing step, the method comprises preprocessing the optical data and the mechanical response data to extract features from the optical data and mechanical response data, and using the extracted features to correlate to the blood pressure.

In embodiments, the method further comprises, during the preprocessing step, separating the data from the optical sensor and the data from the mechanical response sensor into individual heart beats.

In embodiments, the method further comprises, after the separating step, synchronizing the data from the optical sensor and the data from the mechanical response sensor and removing optical sensor data and mechanical response data that does not synchronize.

In embodiments, placing the optical sensor and the mechanical response sensor comprises placing the optical sensor and the mechanical response sensor on opposite lateral surfaces of the person's finger.

In a preferred embodiment, the placing step comprises clamping opposing surfaces of a clip onto the finger. The clamping creates a first pressure against the skin of the person insufficient to occlude arteries under the skin. In a preferred embodiment, the first pressure is less than 30 mmHg.

In embodiments, the computing step is performed by correlating the data to blood pressure using a calibrated model such as a calibrated regression algorithm.

In embodiments, the computing employs machine learning algorithm to correlate the blood pressure to the extracted features.

In embodiments, the computing step is further based on the person's individual characteristics. Examples of a person's individual characteristics include height, weight, and age.

In embodiments, the clamping is carried out by clamping the optical sensor and the mechanical response sensor at a proximal first location along a length of the person's finger (e.g., in the vicinity of the base of the person's finger).

In embodiments, the computing is performed based on the optical sensor data and the mechanical response data from the proximal first location and without repositioning the optical sensor and mechanical response sensor to obtain optical data and mechanical response data at a second location.

Still other descriptions, objects and advantages of the present invention will become apparent from the detailed description to follow, together with the accompanying drawings.

### BRIEF DESCRIPTION OF THE DRAWINGS

FIG. 1 illustrates a blood pressure monitor in accordance with one embodiment of the invention;

FIG. 2 is an enlarged top view of a clip portion of the blood pressure monitor shown in FIG. 1;

FIG. 3 is enlarged view of a sensor incorporated into the clip portion shown in FIG. 2;

FIGS. 4-5 show a top view of the clip portion of FIG. 2 placed on a human finger in a palm up and palm down orientation respectively;

FIG. 6 is a block diagram of the blood pressure monitor in accordance with one embodiment of the invention;

FIG. 7 is a flowchart illustrating a method for computing blood pressure in accordance with an embodiment of the invention;

FIG. 8A is a plot of normalized amplitude versus time for a cuff validated low blood pressure heartbeat of 129/79 mmHg;

FIG. 8B is plot of normalized amplitude versus time for a cuff validated high blood pressure heartbeat of 181/103 mmHg;

FIG. 9 is a plot of normalized piezo amplitude versus time for a blood pressure monitor;

FIG. 10 is a plot of piezo Fourier transformation versus time for the data shown in FIG. 9;

FIG. 11 is an illustration of a procedural setup for an example use of a blood pressure monitor;

FIG. 12 is a plot of systolic pressure arising from a conventional blood pressure monitor versus systolic pressure measured by a blood pressure monitor in accordance with one embodiment of the subject invention; and

FIG. 13 is a plot of diastolic pressure arising from a conventional blood pressure monitor versus diastolic pressure measured by a blood pressure monitor in accordance with one embodiment of the subject invention.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Overview

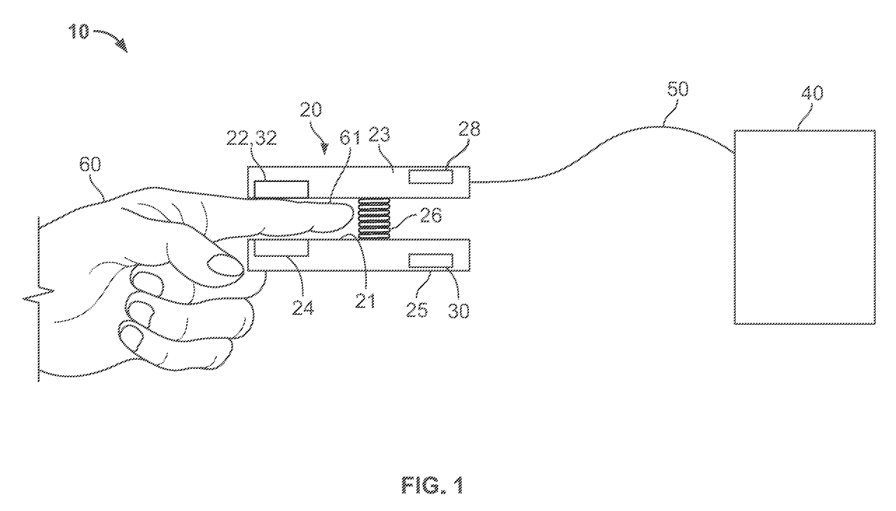

FIG. 1 illustrates a blood pressure monitor 10 in accordance with an embodiment of the invention. The blood pressure monitor 10 is shown having a clip portion 20 and a host portion 40 in communication with the clip portion via serial connector 50. The clip portion 20 is placed on the finger 61 of a user to obtain vascular activity data from arteries within the finger and operates with the host 40 to compute blood pressure values.

The clip 20 can include a number of components such as but not limited to: an optical sensor 22, LED 32, piezoelectric sensor 24, a circuit portion 28 operable with the sensors, and battery pack 30.

The clip can have a wide range of configurations and shapes. The clip 20 shown in FIG. 1 includes a slot or cavity 21 to receive the patient's finger 61. The cavity 21 may be formed by opposing members 23, 25 which are biased or naturally pivot to hold or clamp the finger 61. Although the finger 61 is shown clamped by the clip 20, the bias or force arising from the spring 26 is not sufficient to occlude blood flow within the finger or individual vessels. Desirable springs for use with the clip operate to produce a pressure at the target site of less than 60 mmHg, and in preferred embodiments, less than 30 mmHg. In contrast to occluding type cuffs, the blood monitor system 10 operates by observing the flow of blood and the tissue response to the pulsating flow of blood through the vasculature as described further herein.

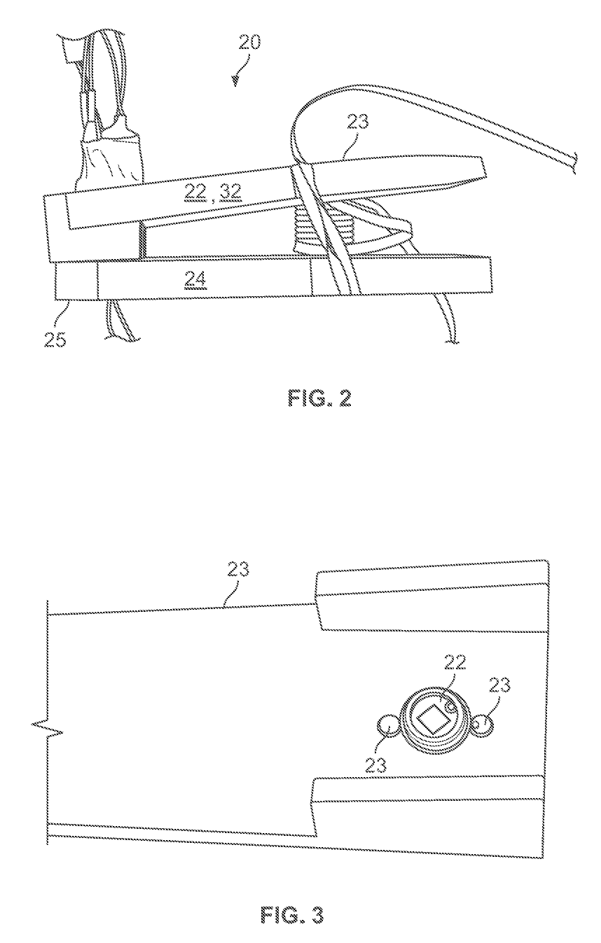

With reference to FIGS. 2-3, sensors 22, 24 are shown incorporated into the opposing members or walls of the clip for contacting or observing activity (e.g., motion, blood flow, etc.) in the finger. FIG. 3 shows optical sensor 22 and adjacent illuminators 23 on an inner surface of the first opposing member 23. Examples of optical sensors include, without limitation, photodiodes, photoresistors, and phototransistors. The optical sensors are typically placed in an area illuminated by a light source. Examples of illuminators include, without limitation, incandescent light, LED, and laser preferably having an emission peak wavelength between 400 nm to 1400 nm.

Orientation

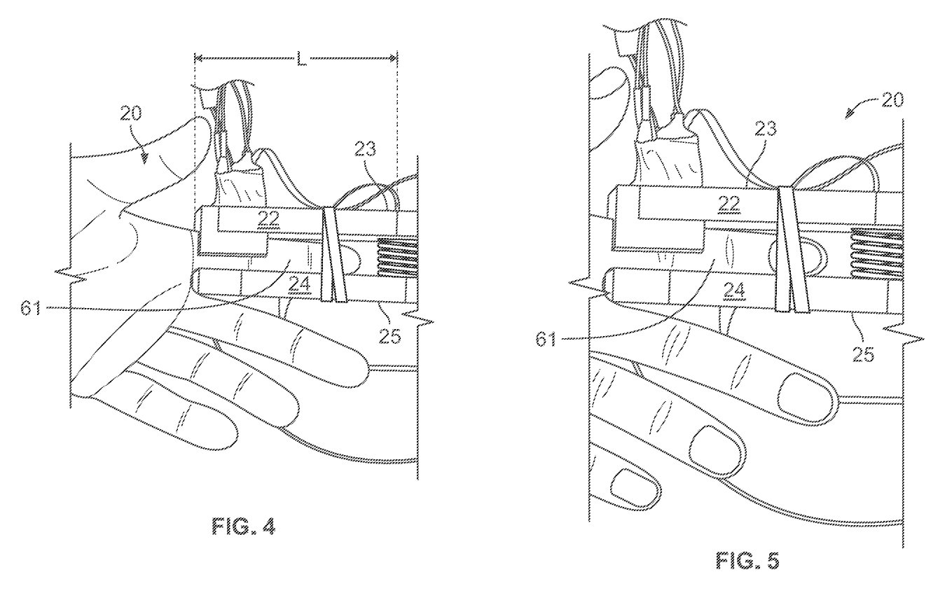

FIGS. 4-5 show examples of the clip 20 placed on a finger 61 of a patient at different suitable orientations. Particularly, with reference to FIGS. 4-5, the inner surfaces of the opposing members 23, 25 are shown contacting lateral surfaces of the index finger in a palm up and palm down orientation respectively.

Additionally, the clip 20 accepts the entire length (L) of the digit to enable the sensors to observe the base portion of the finger near the palm. Because the arteries in the finger tend to run along the sides (lateral surfaces) of the finger and terminate after the first section, the orientations of the clip 20 on the finger shown in FIGS. 4-5 ensure the sensors 22, 24 are closet to the arteries on the sides of the finger, serving to obtain an optimal signal, discussed further herein.

System Architecture

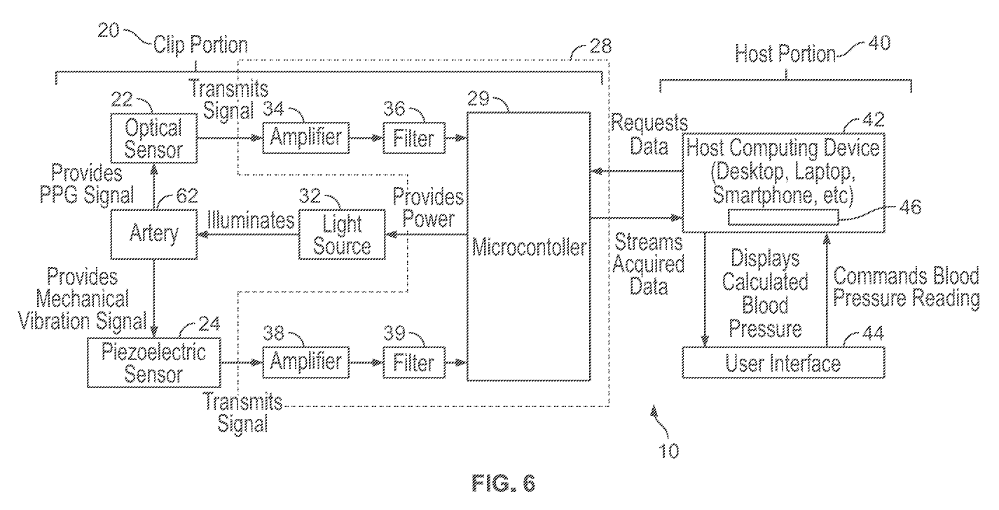

FIG. 6 is a block diagram of a blood pressure monitor system 10 in accordance with an embodiment of the invention. As described herein, the system 10 generally includes a clip portion 20 to obtain the blood pressure data and a host portion 40 to compute the blood pressure based on the data obtained from the clip.

Clip portion 20 is shown including the optical and piezo sensors 22, 24 and a circuit portion 28 for driving the optical sensor and preprocessing the signals from the sensors prior to sending them to the host or central processor 42. The circuit portion 28 is shown including amplifying components 34, 38 and filtering components 36, 39 for the incoming electrical signals from the sensors 22,24 as well as a microcontroller 29 which receives the filtered and amplified signals and streams them to the computing host 42. Examples of components include an Arduino Uno microcontroller, manufactured by Arduino (New York, N.Y.); a Texas Instruments amplifier, part no. LM741CN/NOPB, manufactured by Texas Instruments (Dallas, Tex.), and an Aluminum Electrolytic Capacitor, part no. EEU-FS0J103B, manufactured by Panasonic (Osaka, Japan).

The computing host portion 40 is shown including a computing device 42 and user interface 44. The user interface serves to receive and display information to the patient, user, or physician. Such information may include blood pressure and other metrics as well as individual characteristics of the patient. Examples of the user interface include a display, monitor, tablet, monitor, smart phone, computer, etc.

The computing device 42 includes a processor 46 programmed with software, described further below, to compute blood pressure based on input from the patient and the data being streamed to it from the microcontroller 29. Exemplary computing devices include, without limitation, desktops, laptops, smart phones, tablets, servers, etc.

Without being bound by theory, embodiments of the invention are different from other blood pressure sensors because of the type of information extracted by which it determines blood pressure. Other cuffless blood pressure monitors rely on determining pulse transit time and using that metric to estimate blood pressure. In contrast, embodiments of the invention use multiple sensing modalities (e.g., optical and piezoelectric), to extract features relating to volumetric flow rate of blood, and the effect of the elastic blood vessel on surrounding tissues when blood is being pumped through it. This information along with other biological factors such as height, weight, and age are combined through a model, described further below, to determine the user's blood pressure. Combining the sensed data, and the biologic and other input parameters, sufficient information is gathered to compute blood pressure of the patient or user.

Method for Computing Blood Pressure

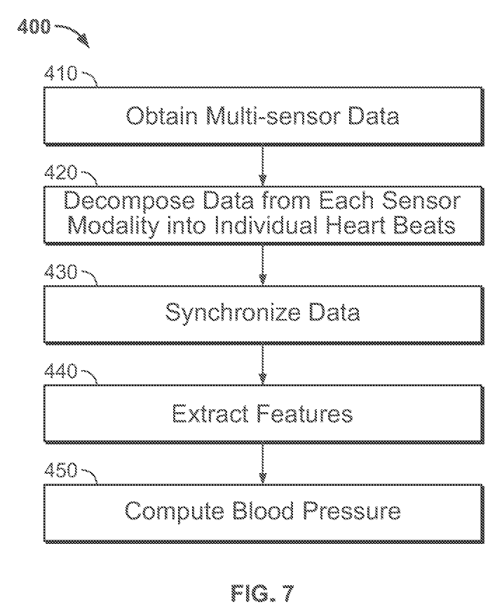

FIG. 7 is a flow chart illustrating a method 400 for computing blood pressure (namely, systolic and diastolic blood pressure) based on the data streamed from the clip and other information input by the user.

Step 410 states to obtain or receive sensor data. For example, when the user requests a blood pressure reading, the blood pressure monitor 10 will stream optical data and mechanical response data to the host for a predetermined duration. The duration may range from 1 to 30 seconds, preferably from 5-20 seconds, and more preferably about 8-12 seconds. In one embodiment, the duration is ten (10) seconds.

Next, with reference to step 420, both the optical data and the mechanical response data are decomposed into the individual heart beats.

Then, with reference to step 430, synchronicity between the optical and mechanical response waveforms will be checked by taking the difference in time that the heart beat separations occur. In embodiments, if there is no congruence between the two waveforms an error will be output to the user that there is not a good signal. Once the heart beats are separated and determined to be congruent across the multiple sensing modalities (e.g., piezo and optical), select features are extracted (step 440) from the waveforms, as described further herein.

Optical Data

Optical data is related to the volume of blood present near the sensor. Recording volume over time defines a photoplethysmogram (PPG). From the PPG, individual heart beats can be isolated and features extracted that relate to blood pressure.

The PPG is composed of a primary wave and a reflective wave with the portion in-between defined as the dicrotic notch. The amount of time between the peak of the primary wave and the peak of the reflective wave is determined by the arterial stiffness. When the blood vessel is deformed by a heartbeat, it stretches and relaxes with hysteresis. This is due to the viscoelastic properties of blood vessels which causes it to elastically reform to its original shape slowly after a stress has been applied (as opposed a Hookean model which reforms instantly after a stress stops being applied).

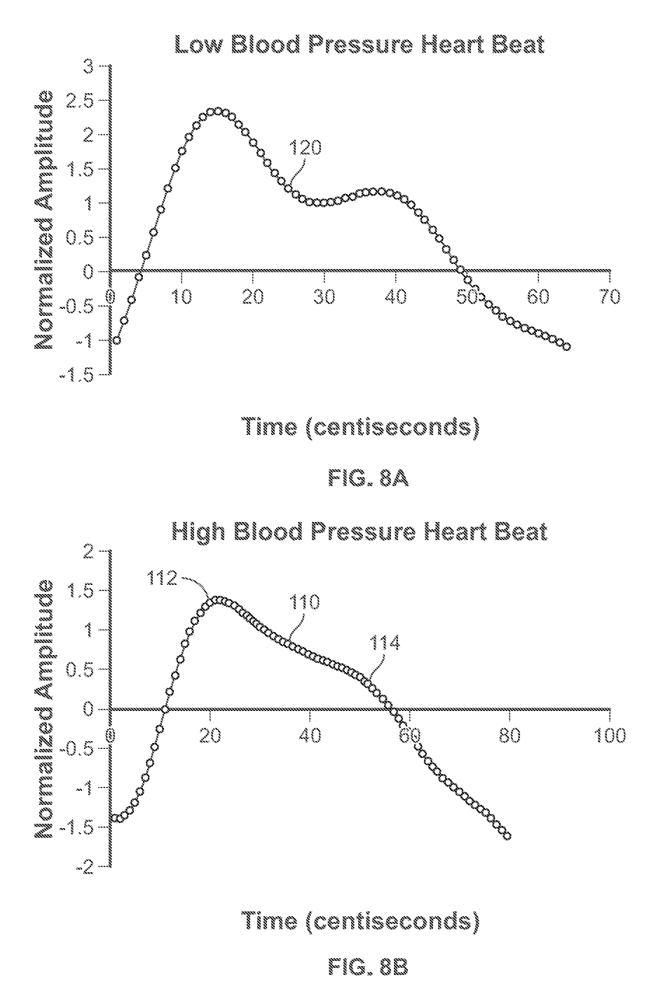

With reference to FIGS. 8A-8B, the higher the pressure applied the greater the magnitude of the hysteresis. In the high blood pressure waveform 110, for example, the vessel does not relax as quickly between the primary wave 112 and the reflective wave 114 as the low pressure waveform 120 (because it has undergone significant enough stress that it cannot finish relaxing before the reflective wave comes). Since this behavior is geometric rather than numeric the features extracted from the waveform represent the geometric characteristics of the waveform. Features are extracted from the derivative, second derivative, and integral of the waveform.

Examples of extracted features include slope, change of slope, and area under the curve. Each feature may be sampled 5-10 times equidistantly. Additionally, the number of features extracted from the optical data may vary. In embodiments, 10-40 features are extracted from the optical data, and more preferably 25-35 features, and in particularly embodiments, about 28-32 features are extracted.

Mechanical Response Data

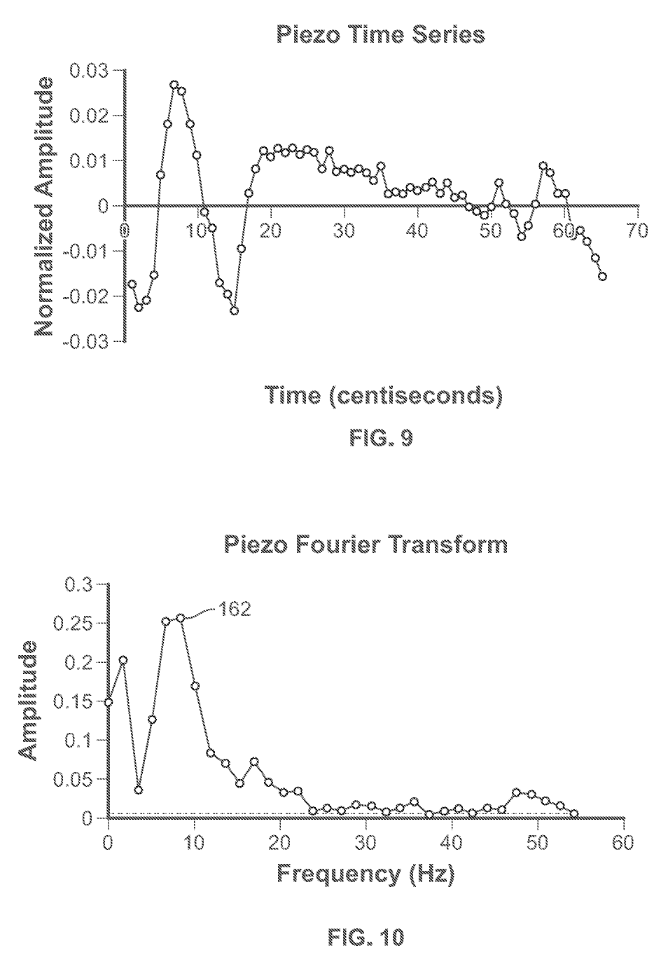

In a preferred embodiment, a piezoelectric sensor is incorporated into the clip to provide mechanical response data of the surrounding tissues. An example of the piezo data is shown in FIG. 9. A purpose of the piezoelectric waveforms is to take into account inter-individual physiological variability in tissues surrounding the blood vessel despite the absence of a direct correlation between blood pressure and the piezoelectric waveforms. If an individual increases their blood pressure artificially (through exercise or other stress) there will not be a change in the piezoelectric data; however, the effect seen from piezoelectric waveforms serves to modulate the previously described relationship between the optical data and blood pressure. This data creates a model that doesn't need to be calibrated on each individual user. The features extracted from the piezoelectric data is in the frequency domain. The tissues around the vessel act as a low pass filter. The feature that relates to the effect of surrounding tissue is the decay rate in the frequency domain which can be seen in FIG. 10 where there is an exponential decay after the max amplitude point 162. In embodiments, only the decay rate is extracted. However, in other embodiments, additional information may be extracted.

Individual Characteristics

As described above, the processor is operable to determine blood pressure based on the data from the sensors. However, in embodiments, the processor combines the data from the sensors with additional information such as individual characteristics to determine blood pressure. In embodiments, a user interface allows a user to input personal characteristics such as her height, weight, and age.

Height has a direct causal link to blood pressure because it contributes to hydrostatic pressure of the blood. Hydrostatic pressure is the pressure exerted on a fluid due to gravity. For this reason, height is also added as a feature by itself into the model.

Height, weight, and age can be used to estimate body fat percentage. Body fat contributes to the lowpass filter effect shown in the piezo data. However, body fat does not necessarily modulate the relationship between the optical data and the blood pressure the same way that collagen or smooth muscle fiber does. Having the body fat percentage will give the model more data to properly account for the effect of surrounding tissues by knowing what amount is fat or another type of tissue.

Combining the individual characteristics and the extracted features from the multi-sensor data provides a plurality of features which are input to a computational model, described below, to compute the blood pressure. In embodiments, a total of 30-40 features are input to the computational model. However, except as recited in the appended claims, more or less features may be fed to the computational model.

Computational Model

Step 450 states to compute the blood pressure. A wide variety of models may be used to compute the blood pressure from the data and extracted features. In embodiments, a multivariate linear model estimates the blood pressure based on the features. An exemplary model is a multivariate ridge regression optionally using Tikhonov regularization.

BP_{sys}=b+Σᵢ₌₀ⁿwᵢxᵢ where b and wᵢ are determined using data from a training set for all i.

BP_{dia}=c+Σⱼ₌₀ⁿwⱼxⱼ where c and wⱼ are determined using data from a training set for all j.

In other embodiments, a non-linear model is employed to estimate the blood pressure based on the features described above. Examples of suitable non-linear models include artificial neural networks and decision trees. In embodiments, the artificial neural network is trained on the data and used as the model for relating various extracted features (such as the extracted features described above) to blood pressure. An artificial neural network model may be preferred to account for the nonlinear relationship between the features and the blood pressure, especially at higher blood pressures over a threshold pressure such as 140 mm Hg systolic, or 110 diastolic.

Function approximation using machine learning (e.g., deep neural nets or ensembled decision trees) is described in various publications such as, for example, Jonas Adler et al, “Solving ill-posed inverse problems using iterative deep neural networks”, Inverse Problems, Volume 33, Issue 12, (2017). The function approximation model can be trained on data gathered through simultaneously recording using the novel blood pressure monitor described herein and an FDA approved sphygmomanometer on a diverse group of subjects. Blood pressure is determined for each separated heartbeat. After blood pressure values are determined for each heart beat they are averaged and then output to the user interface for the operator to read.

### Example

Background: a prototype blood pressure monitor as described above was tested for accuracy. Blood pressure values were obtained through simultaneous measurement using a blood pressure monitor as described above and an FDA approved blood pressure cuff. Because the data being compared is the same dependent variable measured from two different devices, percent root mean squared error was selected to show inaccuracy. Root mean square penalizes small deviations less punitively and large deviations more punitively. This is important because a few large errors are more dangerous than many small errors when making decisions based on data.

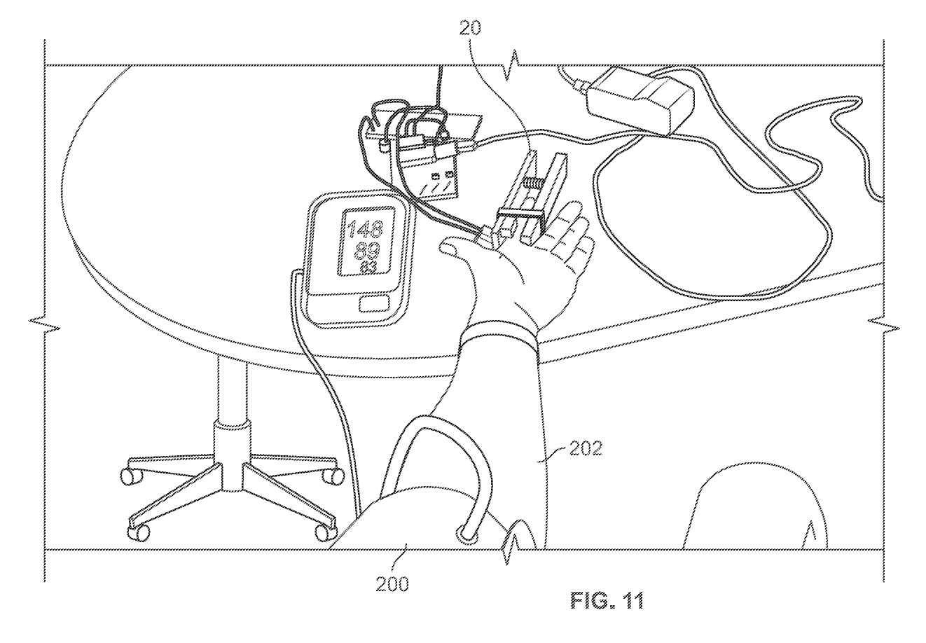

Procedure Setup: with reference to FIG. 11, a conventional blood pressure cuff 200 was placed on an arm 202 and a clip 20 of the new blood pressure monitor as described above was placed on a finger of the same arm. Five (5) seconds of blood flow data was recorded from the blood pressure monitor in accordance with subject invention. After the recording was finished, cuff inflation was quickly commenced. Both systolic and diastolic values were recorded from both the conventional cuff 200 and the clip 20. If the systolic blood pressure was greater than 140 mmHg then it was rejected since the clip is not calibrated to work beyond that value. The above procedural steps were repeated every half hour in different positions to get multiple and a diverse set of blood pressure readings.

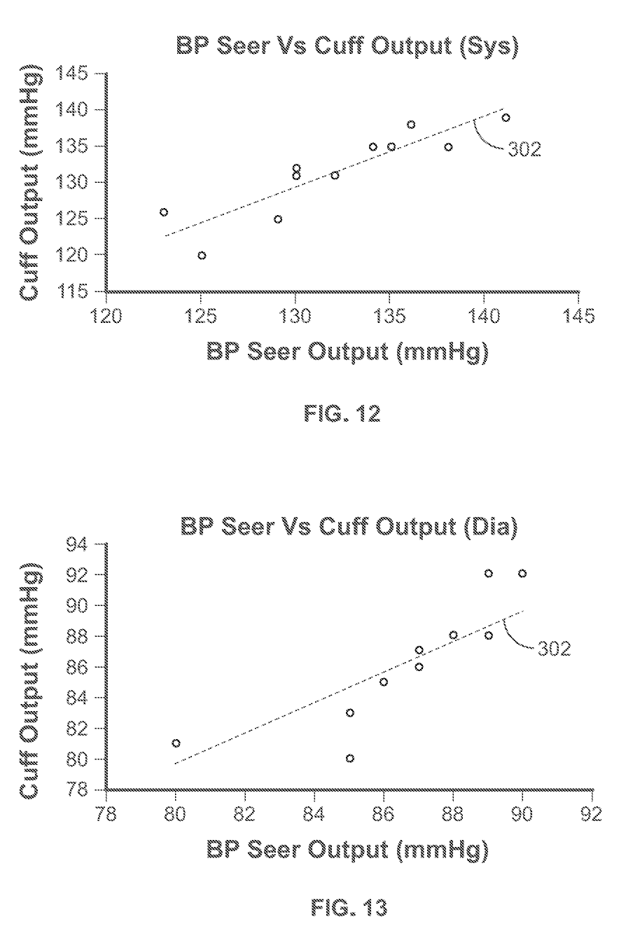

Results: FIG. 12 shows the systolic blood pressure reading from clip plotted against the blood pressure reading from the conventional cuff. The % RMS error=2.05%. The dotted line 302 represents 0 error relationship. FIG. 13 shows the diastolic blood pressure reading from clip plotted against blood pressure reading from the conventional cuff. The % RMS error=2.45%. The dotted line 320 represents 0 error relationship. These results demonstrate that the tested novel blood pressure monitor operates accurately when compared to the gold standard cuff pressure monitor.

### Alternative Embodiments

Although the above described apparatus is described as a clip placed on the finger, it could be configured otherwise. The device could be configured to attach to the ear, wrist or another part of the body where there is an artery that is close to the surface of the skin (in other words not too deep to sense). Examples of other configurations include, without limitation, watch or bracelet, necklace, bracelet, ring, belt whether surrounding the chest, waist, thigh or other area.

It is also to be understood that although the above described clip is shown and described coupled to the host computer via cable, the invention need not be so limited. In embodiments, the host computer, portable reader, or central processor is remote (e.g., cloud based) and the data transmission is achieved wirelessly (e.g., near field or far field) as is known to those of ordinary skill in the art.

Additionally, in other embodiments, additional sensors are combined with the movement and optical sensors. Examples of additional sensors include EKG sensors.

Although a number of embodiments have been disclosed above, it is to be understood that other modifications and variations can be made to the disclosed embodiments without departing from the subject invention. Indeed, any of the components described herein may be combined with one another except where such components are exclusive to one another. Any of the steps described herein may be combined in any combination and sequence except where exclusive to one another.

1. A blood pressure monitor for computing the blood pressure of a patient comprising:
an implement adapted to be placed against the skin of the patient and comprising an optical sensor and a mechanical response sensor; and
a processor operable to compute the blood pressure of the patient based on data from the optical sensor and the mechanical response sensor.

2. The blood pressure monitor of claim 1, wherein the implement is in the shape of a clip and adapted to receive a finger of the patient.

3. The blood pressure monitor of claim 2, wherein the clip comprises a first inner surface and a second inner surface opposite to the first inner surface, and wherein the first inner surface and second inner surface are urged against lateral surfaces of the finger when the clip is placed on the finger.

4. The blood pressure monitor of claim 3, wherein the clip further comprises a spring to bias the inner surfaces to clamp on the finger with a first force less than a threshold force which would occlude blood flow of arteries within the finger.

5. The blood pressure monitor of claim 1, wherein the mechanical response sensor is a piezoelectric sensor.

6. The blood pressure monitor of claim 1, further wherein optical sensor is selected from the group consisting of photodiodes, photoresistors, and phototransistors.

7. The blood pressure monitor of claim 1, wherein the implement further comprises battery.

8. The blood pressure monitor of claim 1, wherein the processor is remote from the implement, and receives data from the optical and mechanical response sensor wirelessly.

9. A method for automatically determining blood pressure of a person comprising:
obtaining optical data by placing an optical sensor against the skin of the person;
obtaining mechanical response data by placing a mechanical response sensor against the skin without occluding a blood vessel under the skin; and
computing on a processor the blood pressure of the person based on the optical data and the mechanical response data.

10. The method of claim 9 further comprising, prior to the computing step, preprocessing the optical data and the mechanical response data to extract features from the optical data and mechanical response data, and using the extracted features to correlate to the blood pressure.

11. The method of claim 10 further comprising, during the preprocessing step, separating the data from the optical sensor and the data from the mechanical response sensor into individual heart beats.

12. The method of claim 11 further comprising, after the separating step, synchronizing the data from the optical sensor and the data from the mechanical response sensor.

13. The method of claim 12 further comprising removing optical sensor data and mechanical response data that does not synchronize.

14. The method of claim 11 wherein the placing the optical sensor and the mechanical response sensor comprises placing the optical sensor and the mechanical response sensor on opposite lateral surfaces of the person's finger.

15. The method of claim 9 wherein the step of obtaining mechanical response data is performed with a piezo sensor.

16. The method of claim 10 wherein the computing employs a machine learning algorithm to correlate the blood pressure to the extracted features.

17. The method of claim 9 wherein the computing step is further based on user input.

18. The method of claim 17 wherein the user input is the person's individual characteristics.

19. The method of claim 18 wherein the person's individual characteristics are characteristics selected from the group consisting of height, weight, and age.

20. The method of claim 9 wherein the placing step comprises clamping opposing surfaces of a clip onto the finger, and wherein the clamping creates a first pressure against the skin of the person insufficient to occlude arteries under the skin.
